(19) United States
(12) Patent Application Publication
(10) Pub. No.: US 2011/0222697 A1
(43) Pub. Date: Sep. 15, 2011

(54) METHOD AND SYSTEM FOR LOCATING A SOUND SOURCE

(75) Inventors: Liang Dong, Shanghai (CN); Maarten Leonardus Christian Brand, Shanghai (CN); Zhongtao Mei, Shanghai (CN)

(73) Assignee: KONINKLIJKE PHILIPS ELECTRONICS N.V., Eindhoven (NL)

(21) Appl. No.: 13/062,864

(22) PCT Filed: Sep. 2, 2009
(86) PCT No.: PCT/IB2009/053819

§ 371 Date: May 27, 2011

(30) Foreign Application Priority Data
Sep. 10, 2008 (CN) 200810212856.X

(51) Int. Cl. A61B 7/04

(52) U.S. Cl. 381/67

(57) ABSTRACT

The invention relates to a method and system for locating a sound source. The system comprises:—a receiving unit (311) for receiving navigating sound signals from at least two navigating sound sensors (21, 22, 23), and receiving a selection instruction comprising a signal segment type corresponding to the sound source, wherein the at least two navigating sound sensors are received in a chest-piece (20),—a selecting unit (312) for selecting a segment from each navigating sound signal according to the signal segment type,—a calculating unit (313) for calculating a difference between the segments selected from the navigating sound signal, and—a generating unit (314) for generating a moving indication signal for guiding moving the chest-piece (20) to the sound source according to the difference.

### FIELD OF THE INVENTION

The invention relates to a method and a system for processing sound signal, particularly, relates a method and a system for locating a sound source by processing a sound signal.

### BACKGROUND OF THE INVENTION

Stethoscope is a very popular diagnosis device used in hospitals and clinics. In the past years, many new technologies have been added to stethoscope to make auscultation more convenient and more reliable. The added new technologies include ambient noise cancellation, auto heart rate counting, auto phonocardiogram (PCG) recording and analysis etc.

Internal sounds of a body may be produced by different organs or even different parts of an organ, which means that the internal sounds are caused by different positions of a body. Taking heart sound as an example: Mitral and tricuspid valves cause heart sound S1; aortic and pulmonary valves cause heart sound S2; and murmurs may originate from valves, chambers or even vessels. Usually, the best place for auscultation is a place which has the highest intensity and the most complete frequency spectrum over an entire body surface. Currently, locating internal sound source is done manually by trained physicians, which requires substantial clinical experiences and great focus.

However, the auscultation skills by manually locating internal sound source are hard to be mastered by non-physicians as it requires knowledge of human anatomy. In addition, the limitations of human ears and perception also influence the localization of the internal sound source of a body. For example, heart sounds S1 and S2 may be close to each other, but both of them are generated by different part of heart. An untrained person may not accurately tell S1 and S2 apart.

### SUMMARY OF THE INVENTION

An object of this invention is to provide a system for locating a sound source conveniently and accurately.

The system for locating a sound source, said system comprises:
a receiving unit for receiving navigating sound signals from at least two navigating sound sensors, and receiving a selection instruction comprising a signal segment type corresponding to the sound source, wherein the at least two navigating sound sensors are received in a chest-piece,
a selecting unit for selecting a segment from each navigating sound signal according to the signal segment type,
a calculating unit for calculating a difference between the segments selected from the navigating sound signals, and
a generating unit for generating a moving indication signal for guiding moving the chest-piece to the sound source according to the difference.

The advantage is that the system can automatically generate a moving indication for accurately locating a sound source, and does not depend on a physician's skills.

The invention also proposes a method corresponding to the system of locating a sound source.

Detailed explanations and other aspects of the invention will be given below.

### DESCRIPTION OF THE DRAWINGS

The above and other objects and features of the present invention will become more apparent from the following detailed description considered in connection with the accompanying drawings, in which:

FIG. 1 depicts a stethoscope in accordance with an embodiment of the invention;

FIG. 2 depicts a chest-piece in accordance with an embodiment of the stethoscope 1 of FIG. 1;

FIG. 3 depicts a system for locating a sound source, in accordance with an embodiment of the stethoscope 1 of FIG. 1;

FIG. 4 depicts a user interface in accordance with an embodiment of the stethoscope 1 of FIG. 1;

FIG. 5 depicts a user interface in accordance with another embodiment of the stethoscope 1 of FIG. 1;

FIG. 6A illustrates a waveform of a sound signal before selecting,

FIG. 6B illustrates a waveform of a sound signal after selecting,

FIG. 7A depicts a waveform of a filtered heart sound signal,

FIG. 7B depicts a waveform of prominent segments,

FIG. 8 is a statistical histogram of intervals between consecutive peak points of the prominent segments,

FIG. 9 is an annotated waveform of a heart sound signal,

FIG. 10 depicts a method of locating a sound source in accordance with an embodiment of the invention.

The same reference numerals are used to denote similar parts throughout the figures.

### DETAILED DESCRIPTION

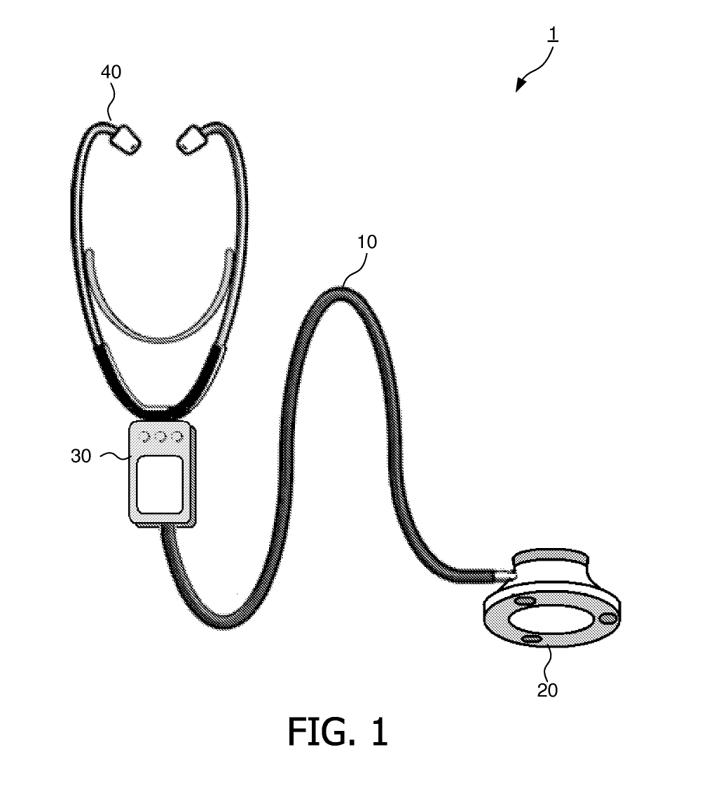

FIG. 1 depicts a stethoscope in accordance with an embodiment of the invention. The stethoscope 1 comprises a chest-piece 20, a control device 30, and a connector 10 for connecting the chest-piece 20 to the control device 30. The stethoscope 1 may also comprises an earphone 40 connecting to the chest-piece 20 through the control device 30 and the connector 10.

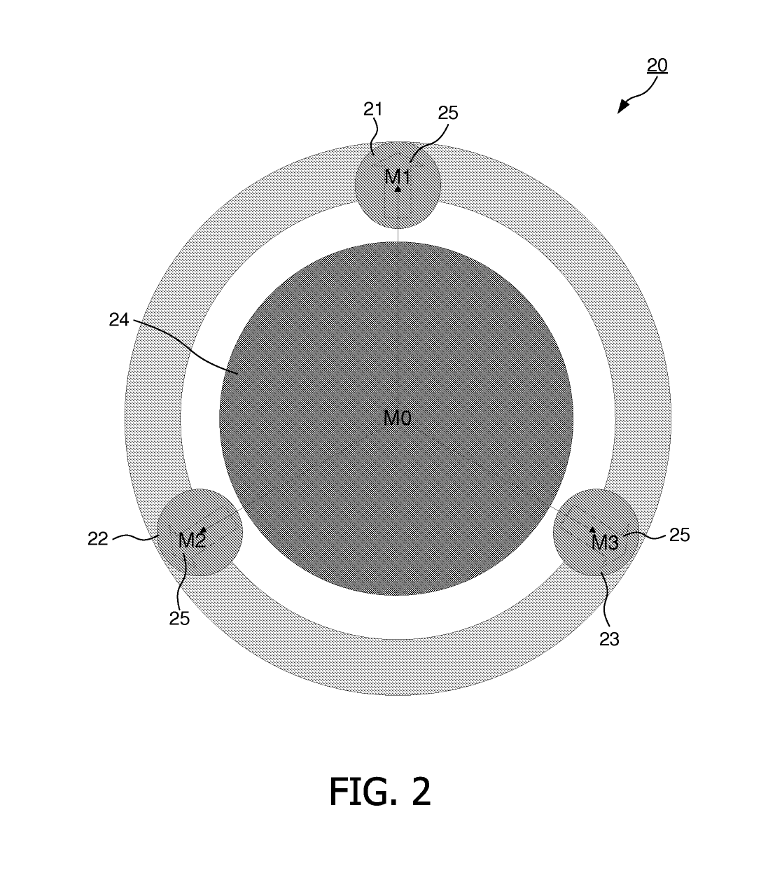

FIG. 2 depicts a chest-piece 20 in accordance with an embodiment of the stethoscope 1 of FIG. 1. The chest-piece 20 comprises a main sound sensor 24 (also shown as M0 in FIG. 2), a first navigating sound sensor 21 (also shown as M1 in FIG. 2), a second navigating sound sensor 22 (also shown as M2 in FIG. 2), and a third navigating sound sensors 23 (also shown as M3 in FIG. 2). The navigating sound sensors 21-23 surround the main sound sensor 24 therein. Preferably, the main sound sensor 24 locates at the central of the chest-piece 20, and the distance from the central of the main sound sensor 24 to each navigating sound sensor is equal, and the angle between every two adjacent navigating sound sensors is equal. The navigating sound sensors 21-23 and the main sound sensor 24 are connected to the control device 30 by the connector 10. The main sound sensor 24 may further connect with the earphone 40 through the control device 30 and the connector 10.

The chest-piece 20 further comprises an indicator 25. The indicator 25 may comprises a plurality of LED lights. Each light corresponds to a navigating sound sensor and is positioned together with the corresponding navigating sound sensor at the same location. The lights can be switched on to guide moving the chest-piece, so as to locate main the sound sensor 24 at a sound source.

Optionally, the indicator 25 may comprise a speaker (not shown in Figures). The speaker is used to generate a voice for guiding moving the chest-piece 20, so as to locate the main sound sensor 24 at a sound source.

The indicators 25 are connected with a circuit (not shown in Figures), and the circuit is used for receiving signal from the control device 30 to control the indicators 25 switching on/off. The circuit can be placed in the chest-piece 20 or the control device 30.

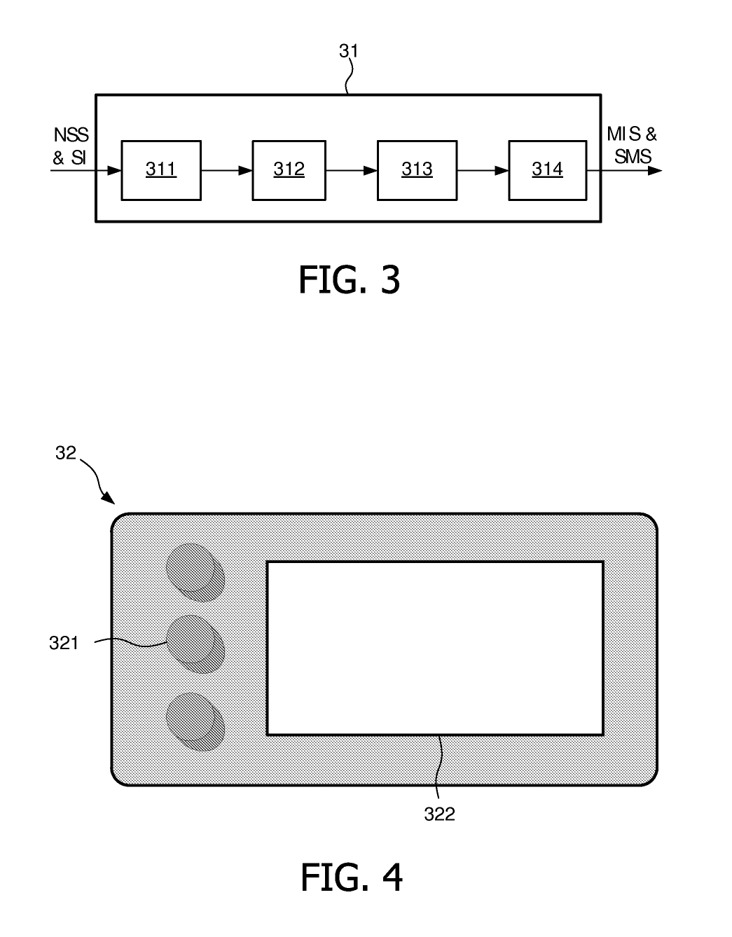

FIG. 3 depicts a system for locating a sound source, in accordance with an embodiment of the stethoscope 1 of FIG. 1. The system 31 comprises a receiving unit 311, a selecting unit 312, a calculating unit 313, and a generating unit 314.

The receiving unit 311 is used for receiving navigating sound signals (shown as NSS in FIG. 3) from the at least two navigating sound sensors 21-23. The receiving unit 311 is also used to receive a selection instruction (shown as SI in FIG. 3), and the selection instruction comprises a signal segment type corresponding to the sound source which is planned to be located by a user. The at least two navigating sound sensors 21-23 are received in the chest-piece 20, and the chest-piece 20 further comprises the main sound sensor 24.

Each navigating sound signal may comprise several segments (or signal segments) which belong to different signal segment types. For example, a heart sound signal detected by the sound sensor may comprise many different signal segment types caused by different sound sources, such as S1 segment, S2 segment, S3 segment, S4 segment, murmurs segment. S1 is caused by the closure of mitral and tricuspid valves; S2 occurs during the closure of aortic and pulmonary valves; S3 is due to the fast ventricular filling during early diastole; S4 occurs as the result of atria contractions displacing blood into the distended ventricular; murmurs may be caused by turbulent blood flow. S1 may be split into M1 caused by Mitral and T1 caused by tricuspid, and S2 may be split into A2 caused by Aortic and P2 caused by Pulmonic valves. S3, S4 and murmurs are usually inaudible and are likely to be associated with cardiovascular diseases.

A user may give a selection instruction for selecting a signal segment type corresponding to a specific sound source to be located, so as to know whether the sound source has disease. For example, the signal segment type to be selected is S1, so the corresponding specific sound source is mitral and tricuspid valves.

The selecting unit 312 is used for selecting a segment from each navigating sound signal according to the signal segment type.

The calculating unit 313 is used for calculating difference between the segments selected from the navigating sound signals. For example, the calculating unit 313 is intended to calculate the difference of the selected segment from the first sound sensor 21 and the selected segment from the second sound sensor 22; calculate the difference of the selected segment from the second sound sensor 22 and the selected segment of the third sound sensor 23; and calculate the difference of the selected segment from the first sound sensor 21 and the selected segment from the third sound sensor 23.

The calculating unit 313 is intended to calculate the difference of TOA (time of arriving) of each segment to the control device 30, since the navigating sound sensors 21-23 are on different places of the chest-piece 20, when the chest-piece 20 is placed on a body, the distances from each navigating sound sensor to the sound source may be different, then the TOA of each selected segment is different.

The calculating unit 313 may be also intended to calculate the difference between the segments by calculating phase difference of the segments. The phase difference can be measured by hardware (such as Field-Programmable Gate Array circuits) or software (such as correlation algorithm).

The generating unit 314 is used to generate a moving indication signal (shown as MIS in FIG. 3) for guiding moving the chest-piece 20 to the sound source according to the difference, so as to locate the main sound sensor 24 at the sound source. The difference may be the TOA difference or the phase difference.

The generating unit 314 may be intended to:
determine a closest navigating sound sensor to the sound source according to the difference between the segments, and
acquire a moving indication signal for guiding moving the chest-piece 20 in a direction of the closest navigating sound sensor to the sound source.

Taking phase difference as an example, if the phase of the segment received from the first navigating sound sensor 21 is bigger than the phase of the segment received from the second navigating sound sensor 22, which means that the distance between the sound source and the second navigating sound sensor 22 is smaller than the distance between the sound source and the first navigating sound sensor 21. The chest-piece 20 should be moved in a direction from the first navigating sound sensor 21 to the second navigating sound sensor 22.

According to the phase difference, the closest navigating sound sensor to the sound source can be determined by comparing the distances between the sound source and the first navigating sound sensor 21, between the sound source and the second navigating sound sensor 22, and between the sound source and the third navigating sound sensor 23. A final moving indication toward the sound source is determined in the direction of the closest navigating sound sensor.

The circuit can receive the moving indication signal from the generating unit 314. The circuit can switch on the indicator 25 to guide moving the chest-piece 20 according to the moving indicator signal. If the indicator 25 is a speaker, the circuit is used to control the indicator 25 to generate a voice for guiding moving the chest-piece 20 according to the moving indication signal, so as to locate the main sound sensor 24 at the sound source; if the indicator 25 comprises a plurality of lights, the circuit is used to control the light, which is corresponding to the closest navigating sound sensor, to be lighted for guiding moving the chest-piece 20, so as to locate the main sound sensor 24 at the sound source.

The generating unit 314 may be used to detect whether the difference of between the segments is lower than a pre-defined threshold. If the difference is lower than the pre-defined threshold, the generating unit 314 may be further intended to generate a stop moving signal (shown as SMS). The circuit can receive the stop moving signal for controlling the indicator 25 to switch off.

FIG. 4 depicts a user interface in accordance with an embodiment of the stethoscope 1 of FIG. 1.

The user interface 32 of the control device 30 comprises a plurality of buttons 321 and an information window 322, such as a display. The information window 322 is used to display a waveform of a sound signal; the buttons 321 are controlled by a user to input a selection instruction for selecting a signal segment type according to attributes reflected by a waveform of the sound signal.

The attributes reflected by a waveform may be a peak, valley, amplitude, duration, frequency etc.

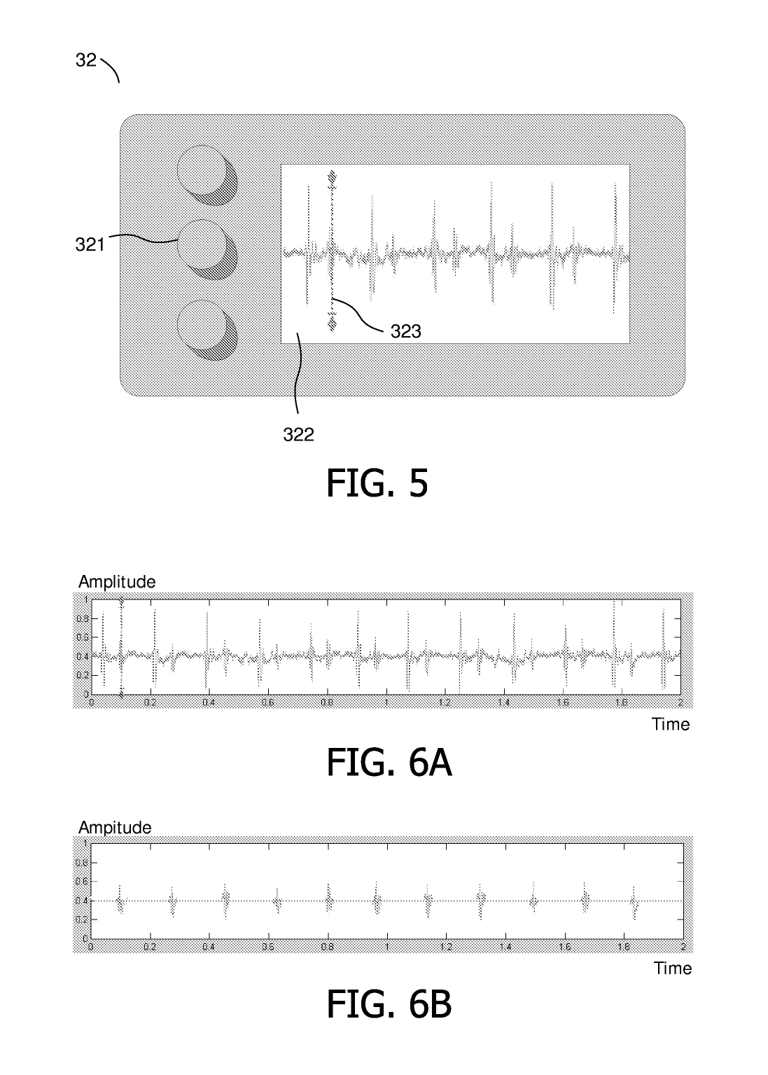

FIG. 5 depicts a user interface in accordance with another embodiment of the stethoscope 1 of FIG. 1. The user interface 32 may comprise a slider 323 for sliding along the waveform to select a specific signal segment type according to the attribute of the waveform.

a further embodiment of the stethoscope 1, the information window 322 may be a touch screen to be touched by a pen or a finger to input a user's selection instruction for selecting a signal segment type from a waveform of a sound signal according to the attribute of the waveform.

According to a user's selection instruction, The selecting unit 312 of the system 31 may be also used to control the information window 322 to show the selected segment and corresponding subsequent segments which is the same type of the selected segment, so that the selected segment is recurrently shown on the information window 32.

Many conventional digital stethoscopes already have the function of selecting a segment from a sound signal, and then only make the selected segment recurrently shown in an information window during receiving the sound signal.

In one embodiment of the invention, the selecting unit 312 may be used in the following way.

FIG. 6A illustrates a waveform of a sound signal before selecting, and FIG. 6B illustrates a waveform of a sound signal after selecting.

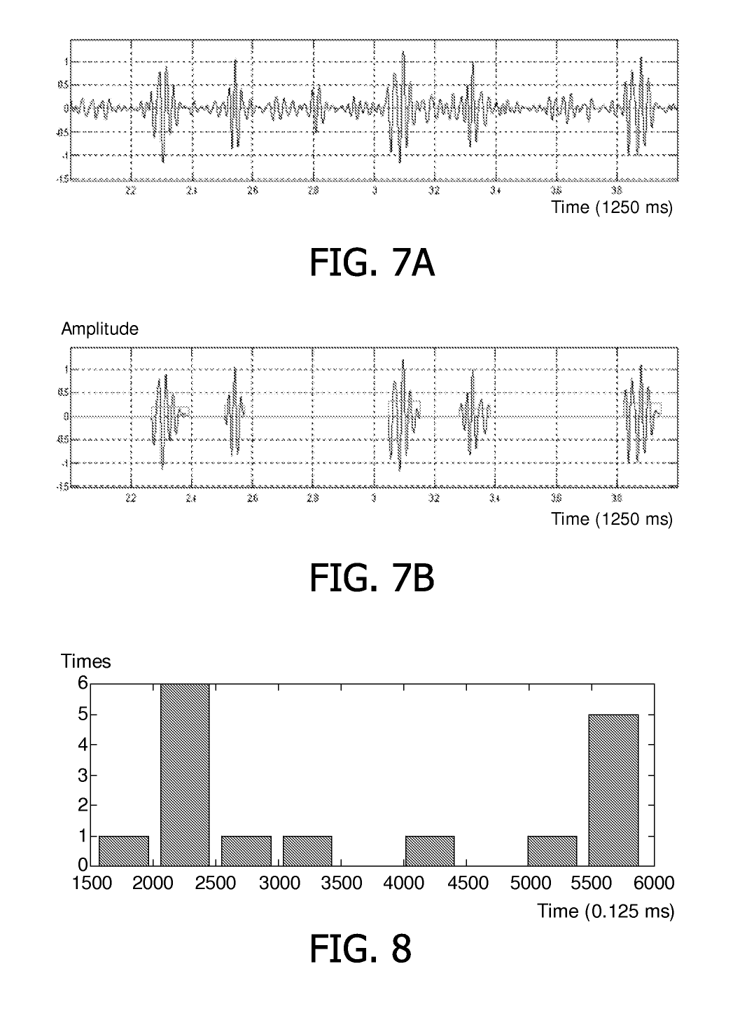

Take heart sound signal as an example, a waveform of a heart sound signal can last at least 5 seconds, so as to support the selecting unit 312 to select a signal segment type according to a user's selection instruction. Supposing the S2 segment is to be selected, the selecting unit 312 may be intended to:
analyze the selection instruction for selecting S2 segment from a heart sound signal.
filter the heart sound signal by a band-pass filter. For example, cut-off frequency 10-100 Hz from the heart sound signal. FIG. 7A depicts a waveform of the filtered heart sound signals.
acquire a plurality of sample points from each segment of the filtered waveform, wherein the waveform is supposed to be divided into several segments.
extract prominent segments which have higher average amplitude variances respectively by computing an average amplitude variance for each segment. For example, the segments with top 5˜10% highest average amplitude variance are referred to as prominent waves. FIG. 7B depicts a waveform for the prominent segments.
measure intervals between consecutive peak points of the prominent segments to form a statistical histogram of the intervals between consecutive peak points of the prominent segments. FIG. 8 is a statistical histogram of the intervals between consecutive peak points of the prominent segments. The statistical histogram may be formed by computing appearance times of each type of interval.
calculate interval between S1 and S2 (called S1-S2 interval in the following) based on the statistical histogram. S1-S2 interval is stable within a short period, e.g. 10 seconds. In the statistical histogram, S1-S2 interval usually appears most frequently. In FIG. 8, the interval between two consecutive peaks within 2000˜2500 sample units (or 0.25˜0.31 second at the sampling rate of 8 KHz) appears 6 times which is the highest appearance frequency and is the S1-S2 interval.
calculate an interval between S2 and S1 based on the statistical histogram. Similarly, the S2-S1 interval is also stable within a short period and is longer than S1-S2 interval. In the statistical histogram, the appearance frequency of S2-S1 interval is only lower than the appearance frequency of S1-S2 interval. In FIG. 8, the interval between two consecutive peaks within 5500˜6000 sample units (or 0.69˜0.75 second at the sampling rate of 8 KHz) appears 5 times, which is only less the appearance frequency of S1-S2 interval, is the S2-S1 interval.
identify S2 segment based on the S1-S2 interval and S2-S1 interval. The S1 segment is identified by entirely searching the prominent segments waveform based on the S1-S2 interval and S2-S1 interval. For example, if the interval between any two consecutive peaks is within the S1-S2 interval as shown in FIG. 8, 2000˜2500 sample units, the segment corresponding to the previous peak is determined as S1, and the subsequent peak is determined as S2.
output a consecutive waveform for the identified S2 segment as shown in FIG. 6B. The consecutive waveform for the identified S2 segments from the at least one navigating sound signals are compared with each other to calculate the difference by the calculating unit 313.

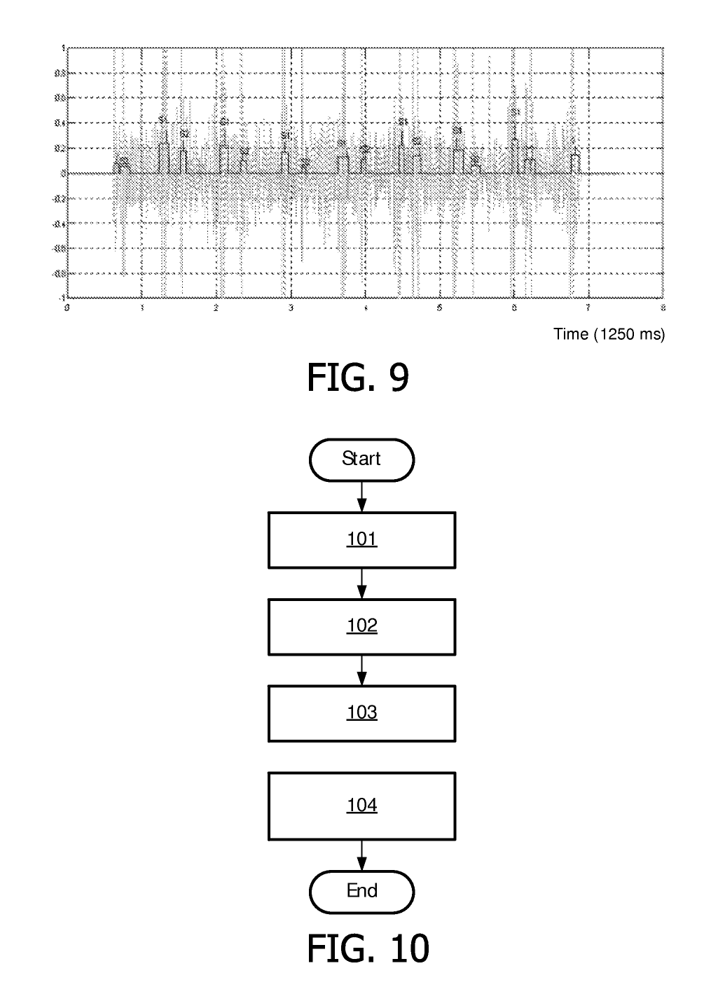

Additionally, the selecting unit 312 can also be used to annotate a sound signal waveform by signal segment type, so that a user can give a selection instruction accurately according to the annotated waveform. During annotating, take a heart sound signal waveform as an example, the selecting unit 312 is used to:
acquire a plurality of sample points from the waveform of the heart sound signal, wherein the waveform is supposed to be divided into several segments.
measure intervals between consecutive peak points of the waveform according to the statistical histogram, as shown in FIG. 8, generated by computing appearance times of each type of interval.
calculate S1-S2 interval based on the statistical histogram. In the statistical histogram, S1-S2 interval usually appears most frequently. The interval between two consecutive peaks within 2000˜2500 sample units (or 0.25˜0.31 second at the sampling rate of 8 KHz) appears 6 times which is the highest appearance frequency and is the S1-S1 interval.
calculate S2-S1 interval based on the statistical histogram. In the statistical histogram, the appearance frequency of S2-S1 interval is only lower than the appearance frequency of S1-S2 interval. The interval between two consecutive peaks within 5500˜6000 sample units (or 0.69˜0.75 second at the sampling rate of 8 KHz) appears 5 times, which is only less the appearance frequency of S1-S2 interval, is the S2-S1 interval.
identify S1 segments and S2 segments based on the S1-S2 interval and S2-S1 interval. The S1 segments are identified by entirely searching the waveform based on the S1-S2 interval and the S2-S1 interval. For example, if the interval between any two consecutive peaks is within the learned S1-S2 interval as shown in FIG. 8, 2000˜2500 sample units, the segment corresponding to the previous peak is determined as S1, and the subsequent peak is determined as S2.
annotate the S1 segments and the S2 segments on the waveform of the heart sound signal. FIG. 9 depicts a waveform for the annotated heart sound signal. The non-recurrent segments, which are treated as noise, are also determined and indicated as “?” in FIG. 9.

Furthermore, if a split exists in S1 signal or/and S2 signal, the split S1 signal and S2 signal may be annotated by analyzing the peak of the S1 signal and S2 signal. For example, a split S1 signal is marked as M1 and T1 (not shown in FIG. 9).

FIG. 10 depicts a method of locating a sound source in accordance with an embodiment of the invention. The method comprises a receiving step 101, a selecting step 102, a calculating step 103, and a generating step 104.

The receiving step 101 is intended to receive navigating sound signals from the at least two navigating sound sensors 21-23. The receiving step 101 is also intended to receive a selection instruction, and the selection instruction comprises a signal segment type corresponding to the sound source which is planned to be located by a user. The at least two navigating sound sensors 21-23 are allocated in a chest-piece 20, and the chest-piece further comprises a main sound sensor 24.

Each navigating sound signal may comprise several segments (or signal segments) which belong to different signal segment types. For example, a heart sound signal detected by the sound sensor may comprise many different signal segment types, such as S1 segment, S2 segment, S3 segment, S4 segment, murmurs segment. S1 is caused by the closure of mitral and tricuspid valves; S2 occurs during the closure of aortic and pulmonary valves; S3 is due to the fast ventricular filling during early diastole; S4 occurs as the result of atria contractions displacing blood into the distended ventricular; murmurs may be caused by turbulent blood flow. S1 may be split into M1 caused by Mitral and T1 caused by tricuspid, and S2 may be split into A2 caused by Aortic and P2 caused by Pulmonic valves. S3, S4 and murmurs are usually inaudible and are likely to be associated with cardiovascular diseases.

A user may give a selection instruction for selecting a signal segment type corresponding to a specific sound source, so as to know whether the sound source has disease, and the signal segment type selected by the user. For example, the sound signal type to be selected is S1, so the corresponding specific sound source is mitral and tricuspid valves.

The selecting step 102 is intended to select a segment from each navigating sound signal according to the signal segment type.

The calculating step 103 is intended to calculate difference between the segments selected from the navigating sound signals. For example, the calculating step 103 is intended to calculate the difference of the selected segment from the first sound sensor 21 and the selected segment from the second sound sensor 22; calculate the difference of the selected segment from the second sound sensor 22 and the selected segment of the third sound sensor 23; and calculate the difference of the selected segment from the first sound sensor 21 and the selected segment from the third sound sensor 23.

The calculating step 103 may be also intended to calculate the difference between the segments by calculating phase difference of the segments. The phase difference can be measured by hardware (such as Field-Programmable Gate Array circuits) or software (such as correlation algorithm).

The generating step 104 is intended to generate a moving indication signal (shown as MIS in FIG. 3) for guiding moving the chest-piece 20 to the sound source according to the difference, so as to locate the main sound sensor 24 to the sound source. The difference may be the TOA difference or the phase difference.

The generating step 104 may be intended to:
determine a closest navigating sound sensor to the sound source according to the difference between the segments, and
acquire a moving indication signal for guiding moving the chest-piece 20 in a direction of the closest navigating sound sensor to the sound source.

The generating step 104 may be intended to detect whether the difference of between the segments is lower than a pre-defined threshold. If the difference is lower than the pre-defined threshold, the generating step 104 may be further intended to generate a stop moving signal (shown as SMS). The circuit can receive the stop moving signal for controlling the indicator 25 to switch off.

Many conventional digital stethoscopes already have the function of selecting a segment of a sound signal, and then only make the selected segment recurrently shown in an information window during receiving the sound signal.

Supposing the S2 segment is to be selected from a heart sound signal as shown in FIG. 6A. In one embodiment of the invention, the selecting step 102 may be intended to:
analyze the selection instruction for selecting S2 segment from a heart sound signal.
filter the heart sound signal by a band-pass filter. For example, cut-off frequency 10-100 Hz from the heart sound signals. The filtered heart sound signal is shown in FIG. 7A.
acquire a plurality of sample points from each segment of the filtered waveform, wherein the waveform is supposed to be divided into several segments.
extract prominent segments which have higher average amplitude variances respectively by computing an average amplitude variance for each segment. For example, the segments with top 5˜10% highest average amplitude variance are referred to as prominent waves. The extracted prominent segments waveform is shown in FIG. 7B.
measure intervals between consecutive peak points of the prominent segments to form a statistical histogram of the intervals between consecutive peak points of the prominent segments. The statistical histogram as shown in FIG. 8 may be formed by computing appearance times of each type of interval.
calculate interval between S1 and S2 (called S1-S2 interval in the following) based on a statistical histogram. S1-S2 interval is stable within a short period, e.g. 10 seconds. In the statistical histogram, S1-S2 interval usually appears most frequently. The interval between two consecutive peaks within 2000˜2500 sample units (or 0.25˜0.31 second at the sampling rate of 8 KHz) appears 6 times which is the highest appearance frequency and is the S1-S1 interval.
calculate an interval between S2 and S1 based on the statistical histogram. Similarly, the S2-S1 interval is also stable within a short period and is longer than S1-S2 interval. In the statistical histogram, the appearance frequency of S2-S1 interval is only lower than the appearance frequency of S1-S2 interval. The interval between two consecutive peaks within 5500˜6000 sample units (or 0.69˜0.75 second at the sampling rate of 8 KHz) appears 5 times, which is only less the appearance frequency of S1-S2 interval, is the S2-S1 interval.
identify S2 segment based on the S1-S2 interval and S2-S1 interval. The S1 segment is identified by entirely searching the prominent segments waveform based on the S1-S2 interval and S2-S1 interval. For example, if the interval between any two consecutive peaks is within the S1-S2 interval as shown in FIG. 8, 2000˜2500 sample units, the segment corresponding to the previous peak is determined as S1, and the subsequent peak is determined as S2.
output a consecutive waveform for the identified S2 segment as shown in FIG. 6B. The consecutive waveform for the identified S2 segments from the at least one navigating sound signals are compared with each other to calculate the difference by the calculating unit 313.

It should be noted that the above-mentioned embodiments illustrate rather than limit the invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. The invention can be implemented by unit of hardware comprising several distinct elements and by unit of a programmed computer. In the system claims enumerating several units, several of these units can be embodied by one and the same item of hardware or software. The usage of the words first, second and third, et cetera, does not indicate any ordering. These words are to be interpreted as names.

1. A system (31) for locating a sound source, said system comprising:
a receiving unit (311) for receiving navigating sound signals from at least two navigating sound sensors (21, 22, 23), and receiving a selection instruction comprising a signal segment type corresponding to the sound source, wherein the at least two navigating sound sensors are received in a chest-piece (20),
a selecting unit (312) for selecting a segment from each navigating sound signal according to the signal segment type,
a calculating unit (313) for calculating a difference between the segments selected from the navigating sound signals, and
a generating unit (314) for generating a moving indication signal for guiding moving the chest-piece (20) to the sound source according to the difference.

2. A system as claimed in claim 1, wherein the calculating unit (313) is intended to calculate the difference between phases of the segments, or to calculate the difference between arriving times of the segments.

3. A system as claimed in claim 1, wherein the generating unit (314) is intended to:
determine a closest navigating sound sensor to the sound source, according to the difference between the segments, and
acquire the moving indication signal for guiding moving the chest-piece (20) in a direction of said closest navigating sound sensor.

4. A system as claimed in claim 3, where the generating unit (314) is intended to determine the closest navigating sound sensor to the sound source by comparing the distance between the sound source and the navigating sound sensors (21, 22, 23).

5. A system according to claim 1, wherein the generating unit (314) is further intended to generate a stop moving signal for guiding to stop moving the chest-piece (20), if the difference of the segments is lower than a pre-defined threshold.

6. A stethoscope comprising the system (31) for locating a sound source as claimed in claim 1.

7. A stethoscope as claimed in claim 6, further comprising a chest-piece (20), a control device (30) integrated the system (31) therein, and a connector 10 for connecting the chest-piece (20) to the control device (30).

8. A chest-piece (20) connected to the system (31) as claimed in claim 1, comprising a circuit and an indicator (25), wherein the circuit is used for receiving the moving indication signal and the stop moving signal to control the indicator (25) switching on/off, so as to guide moving/stopping moving the chest-piece (20).

9. A chest-piece (20) as claimed in claim 8, wherein the indicator (25) comprises at least two lights corresponding to the at least two navigating sound sensors (21, 22, 23), when the moving indication indicates to move along a direction of a navigating sound sensor, the light corresponding to the navigating sound sensor is switched on for guiding moving the chest-piece (20), and when the circuit receives the stop moving signal, the light is switched off to indicate stopping moving the chest-piece (20).

10. A chest-piece as claimed in claim 8, wherein the indicator (25) comprises a speaker, when the circuit receives the moving indication signal/the stop moving signal, the speaker voices to guide moving/stopping moving the chest-piece (20).

11. A method of locating a sound source, said method comprising the steps of:
receiving (101) navigating sound signals from at least two navigating sound sensors (21, 22, 23), and receiving a selection instruction comprising a signal segment type corresponding to the sound source, wherein the at least two navigating sound sensors are received in a chest-piece (20),
selecting (102) a segment from each navigating sound signal according to the signal segment type,
calculating (103) a difference between the segments selected from the navigating sound signals, and
generating (104) a moving indication signal for guiding moving the chest-piece (20) to the sound source according to the difference.

12. A method as claimed in claim 11, wherein the calculating step (103) is intended to calculate the difference between the phases of the segments, or to calculate the difference between the arriving times of the segments.

13. A method as claimed in claim 11, wherein the generating step (104) is intended to:
determine a closest navigating sound sensor to the sound source according to the difference between the segments, and
acquire the moving indication signal for guiding moving the chest-piece (20) in a direction of the closest navigating sound sensor.

14. A method as claimed in claim 13, where the generating step (104) is further intended to determine the closest navigating sound sensor to the sound source by comparing the distance between the sound source and the navigating sound sensors (21, 22, 23).

15. A method according to claim 11, wherein the generating step (104) is further intended to generate a stop moving signal for guiding to stop moving the chest-piece (20), if the difference of the segments is lower than a pre-defined threshold.
